(19) United States
(12) Patent Application Publication
(10) Pub. No.: US 2021/0343394 A1
(43) Pub. Date: Nov. 4, 2021

(54) MENTAL WELLNESS PLATFORM USING PERSONALIZED DREAM ANALYSIS

(71) Applicant: Sansan Fibich, New York, NY (US)

(72) Inventor: Sansan Fibich, New York, NY (US)

(21) Appl. No.: 17/126,092

(22) Filed: Dec. 18, 2020

Related U.S. Application Data
(60) Provisional application No. 62/949,896, filed on Dec. 18, 2019.

(51) Int. Cl. G16H 20/70; G16H 10/20; G16H 50/20; G16H 80/00; G16H 40/20; G06N 20/00; G10L 15/26; G10L 15/22; A61B 5/00

(52) U.S. Cl. CPC G16H 20/70; G16H 10/20; G16H 50/20; G16H 80/00; G16H 40/20; G16H 50/70; G10L 15/26; G10L 15/22; A61B 5/4815; A61B 5/4812; G06N 20/00

(57) ABSTRACT

A dream analysis platform having a processor and a memory operatively coupled to the processor, the memory having stored thereon instructions for realizing aspects of the platform, including a plurality of symbols known to appear in dreams, each symbol stored in association with at least one corresponding possible meaning, and/or a subject's cognitive association with said symbol; a user interface comprising a plurality of screens to be shown on a user device having a display; an alarm configurable to awaken a user when the user is asleep and expected to be dreaming or at a time most convenient to the user; and a dream prompting engine configured to prompt a user to record a dream that they recalled when the alarm woke the user.

### FIELD OF THE INVENTION

The present invention and its embodiments relate to gathering information of dreams experienced by a subject, and helping the subject analyze and understand their dreams.

### BACKGROUND OF THE EMBODIMENTS

Virtually everyone sleeps, and everyone has dreams while sleeping. Many people believe, and much scientific research supports their belief, that their dreams have meaning relevant to their mental, emotional, and waking life, but often the meaning is obscured by symbolic imagery. Dream analysis is the process of assigning meaning to dream symbols, imagery, and emotional content. In some ancient societies, such as ancient Egypt for example, dreams were sometimes considered a supernatural communication whose message could be ascertained only by people with a divine gift of analysis. The modern view (supported by evidence} is the relevance of dreams is in the point that dream content primarily deals with the dreamer's emotional challenges related to experiences they are presently going through. This is largely known as the Continuity Theory of dreams.

Systematic research demonstrates the dream is a unique psychodynamically informative instrument for evaluating the subjective correlates of brain activity during REM sleep. These include feelings, percepts, memories, wishes, fantasies, impulses, conflicts, and defenses, as well as images of self and others. Dreams may serve as the initial indicators of transference, resistance, impending crisis, acting-out, conflict resolution, and decision-making. (The Journal of Psychotherapy Practice and Research 2001; 10:223-230)

In modern times, many people believe dreams may be particularly meaningful. For example, it has been reported that some people would be more likely to cancel a looming trip if they dreamt of their plane crashing than if a Federal warning was issued. (Morewedge, Norton, “When dreaming is believing: The analysis of dreams′, J Pers Soc Psychol. 2009 February}.

Modern dream analysis finds its inception in a book called The Analysis of Dreams published in 1900 by Sigmund Freud. This book describes Freud's early theories of the subconscious with respect to dream analysis. In Freud's view, dreams are formed as the result of two opposing mental processes. The first is driven by the subconscious, which results in dreams with imagery that expresses desires. The other is driven by censorship borne of social convention, and results in imagery that distorts those expressions. As such, dream imagery is unpredictable, inconsistent, and subject to interpretation. Accordingly, the meaning of dreams is often difficult to determine.

In addition, of course most people do not awaken while dreaming, and do not remember their dreams unless they are particularly disturbing. Accordingly, at least some of the potential conscious benefit of dreams may be lost.

### SUMMARY OF THE EMBODIMENTS

A dream analysis platform comprising a processor and a memory operatively coupled to the processor, the memory having stored thereon instructions for realizing aspects of the platform, including a plurality of symbols known to appear in dreams, each symbol stored in association with at least one corresponding possible meaning, and/or a subject's cognitive association with said symbol; a user interface comprising a plurality of screens to be shown on a user device having a display; an alarm configurable to awaken a user when the user is asleep and expected to be dreaming or at a time most convenient to the user; a dream prompting engine configured to prompt a user to record a dream that they recalled when the alarm woke the user, a dream recording engine configured to record user input describing the dream they were having when awakened; a voice-to-text transcribing engine; a conversational Artificial Intelligence (AI) agent configured to prompt the user with questions and suggestions regarding elements from the user's initial input and/or elements from past inputs; a mood tracking engine collecting data on the user's mood; a thematic tagging engine that tags dream content and user input according to psychological persona and cognitive behaviors principles; a dream analysis engine that tags and analyzes the dream from inputs including the plurality of symbols and the recording of the dream and inputs from subsequent prompts; a matching engine that personalizes relaxation and resolution techniques tailored to said tagging and other pertinent user psychographics; a services marketplace of human coaches and a matching engine to match the user to the appropriate coach according to current and previous user data analyses, and an interface facilitating small group and/or one-on-one live video coaching sessions; and a “Big Data” database that analyzes anonymized data, and stores for future use, potentially in clinical or healthcare settings. In embodiments, the analysis engine utilizes artificial intelligence (AI), machine learning (ML), and natural language processing (NLP) technologies.

It is to be understood that both the foregoing general description and the following detailed description are exemplary and explanatory and are intended to provide further explanation of the invention as claimed.

### BRIEF DESCRIPTION OF THE DRAWINGS

The accompanying drawings are included to provide an understanding of the invention. The drawings illustrate disclosed embodiments and/or aspects and, together with the description, explain principles of the invention, the scope of which is determined by the claims.

FIG. 1A shows an exemplary screenshot including an introductory screen, splash screens, and login screens;

FIG. 1B shows an exemplary screenshot including an introductory screen, splash screens, and login screens.

FIG. 1C shows an exemplary screenshot including an introductory screen, splash screens, and login screens.

FIG. 1D shows an exemplary screenshot including an introductory screen, splash screens, and login screens.

FIG. 1E shows an exemplary screenshot including an introductory screen, splash screens, and login screens.

FIG. 1F shows an exemplary screenshot including an introductory screen, splash screens, and login screens.

FIG. 2A shows an exemplary screenshot including screens to gather user profile information;

FIG. 2B shows an exemplary screenshot including screens to gather user information.

FIG. 2C shows an exemplary screenshot including screens to gather user information.

FIG. 2D shows an exemplary screenshot including screens to gather user information.

FIG. 2E shows an exemplary screenshot including screens to gather user information.

FIG. 2F shows an exemplary screenshot including screens to gather user information.

FIG. 2G shows an exemplary screenshot including screens to gather user information.

FIG. 2H shows an exemplary screenshot including screens to gather user information.

FIG. 2I shows an exemplary screenshot including screens to gather user information.

FIG. 2J shows an exemplary screenshot including screens to gather user information.

FIG. 2K shows an exemplary screenshot including screens to gather user information.

FIG. 2L shows an exemplary screenshot including screens to gather user information.

FIG. 3A shows an exemplary screenshot including screens for setting a dreamtime alarm, gathering dream information, and providing suggested analyses of salient dream elements and of the dream in general.

FIG. 3B shows an exemplary screenshot including screens for setting a dreamtime alarm, gathering dream information, and providing suggested analyses of salient dream elements and of the dream in general.

FIG. 3C shows an exemplary screenshot including screens for setting a dreamtime alarm, gathering dream information, and providing suggested analyses of salient dream elements and of the dream in general.

FIG. 3D shows an exemplary screenshot including screens for setting a dreamtime alarm, gathering dream information, and providing suggested analyses of salient dream elements and of the dream in general.

FIG. 3E shows an exemplary screenshot including screens for setting a dreamtime alarm, gathering dream information, and providing suggested analyses of salient dream elements and of the dream in general.

FIG. 3F shows an exemplary screenshot including screens for setting a dreamtime alarm, gathering dream information, and providing suggested analyses of salient dream elements and of the dream in general.

FIG. 3G shows an exemplary screenshot including screens for setting a dreamtime alarm, gathering dream information, and providing suggested analyses of salient dream elements and of the dream in general.

FIG. 3H shows an exemplary screenshot including screens for setting a dreamtime alarm, gathering dream information, and providing suggested analyses of salient dream elements and of the dream in general.

FIG. 3I shows an exemplary screenshot including screens for setting a dreamtime alarm, gathering dream information, and providing suggested analyses of salient dream elements and of the dream in general.

FIG. 3J shows an exemplary screenshot including screens for setting a dreamtime alarm, gathering dream information, and providing suggested analyses of salient dream elements and of the dream in general.

FIG. 3K shows an exemplary screenshot including screens for setting a dreamtime alarm, gathering dream information, and providing suggested analyses of salient dream elements and of the dream in general.

FIG. 3L shows an exemplary screenshot including screens for setting a dreamtime alarm, gathering dream information, and providing suggested analyses of salient dream elements and of the dream in general.

FIG. 3M shows an exemplary screenshot including screens for setting a dreamtime alarm, gathering dream information, and providing suggested analyses of salient dream elements and of the dream in general.

FIG. 3N shows an exemplary screenshot including screens for setting a dreamtime alarm, gathering dream information, and providing suggested analyses of salient dream elements and of the dream in general.

FIG. 3O shows an exemplary screenshot including screens for setting a dreamtime alarm, gathering dream information, and providing suggested analyses of salient dream elements and of the dream in general.

FIG. 3P shows an exemplary screenshot including screens for setting a dreamtime alarm, gathering dream information, and providing suggested analyses of salient dream elements and of the dream in general.

FIG. 3Q shows an exemplary screenshot including screens for setting a dreamtime alarm, gathering dream information, and providing suggested analyses of salient dream elements and of the dream in general.

FIG. 4A shows an exemplary screenshot of a screen for the alarm being activated at the previously selected time, prompting the user to begin awaking and select to either record the dream or, alternately, use the mood tracker to enter their mood.

FIG. 4B shows an exemplary screenshot of a screen for the user to record the dream.

FIG. 5A shows an exemplary screenshot of a screen for the recorded dream to display as the conversational agent prompts the user with questions regarding a dream element and suggesting possible meaning for the user to select from.

FIG. 5B shows an exemplary screenshot of a screen for the recorded dream, as well as the user's input from FIG. 5A to display as the conversational agent prompts the user with questions regarding an additional dream element and suggesting possible meaning for the user to select from.

FIG. 5C shows an exemplary screenshot of a screen for prompting the user to type in any current life event or circumstances that seems related to the dream.

FIG. 5D shows an exemplary screenshot of a screen displaying an animated symbol depicting the engine's analysis processing.

FIG. 5E shows an exemplary screenshot of a screen for the dream text, the user's input from FIGS. 5A-5B, and additional similar inputs to display, as the conversational agent conveys the dream's analysis to the user in a conversational/textual manner.

FIG. 6A shows an exemplary screenshot of a screen displaying the top tier of the mood tracker as a color-coded diagram for the user to select their current mood.

FIG. 6B shows an exemplary screenshot of a screen displaying the second tier of the mood tracker as a color-coded diagram displaying a number of sub-moods related to the user's first tier pick, for the user to select their current sub-mood.

FIG. 7A shows an exemplary screenshot of a screen displaying the number of the user's entries for the time period set by the engine as an encouraging “badge” for the user to see the points they earn towards a bonus reward.

FIG. 7B shows an exemplary screenshot of a screen displaying the latest entry as reaching the number of entries for the time period required as an award “badge” celebrating the user's milestone.

FIG. 8 shows exemplary screenshot of a screen for the awarded feature: A personalized map, diagram, or infographic displaying analyses as psychological thematic tagging in relation to the dates dreams were inputted and to the user's inputted correlating life events.

FIG. 9A shows an exemplary example of a screenshot of the system.

FIG. 9B shows an exemplary example of a screenshot of the system

### DETAILED DESCRIPTION

It is to be understood that the figures and descriptions provided here may have been simplified to illustrate aspects that are relevant for a clear understanding of the herein described processes, machines, manufactures, and/or compositions of matter, while eliminating, for the purpose of clarity, other aspects that may be found in typical devices, systems, and methods. Those of ordinary skill in the pertinent art may recognize that other elements and/or steps may be desirable and/or necessary to implement the devices, systems, and methods described herein.

Because such elements and steps are well known in the art, and because they do not facilitate a better understanding of the present disclosure, a discussion of such elements and steps may not be provided herein. However, the present disclosure is deemed to inherently include all such elements, variations, and modifications to the described aspects that would be known to those of ordinary skill in the pertinent art.

It will be readily understood that the components of the present invention, as generally described and illustrated in the figures herein, may be realized in a variety of different configurations. Thus, the following detailed description of the embodiments of a method, apparatus, and system, as represented in the attached figures, is not intended to limit the scope of the invention as claimed, but is merely representative of selected illustrative embodiments of the invention. The usage of the phrases “example embodiments”, “some embodiments”, or other similar language, throughout this specification refers to the fact that a particular feature, structure, or characteristic described in connection with the embodiment may be included in at least one embodiment of the present invention, and do not necessarily all refer to the same group of embodiments.

A variety of embodiments will now be described. These embodiments are provided as teaching examples and should not be analyzed to limit the scope of the invention. Although specific details of the embodiments are presented, these embodiments may be modified by changing, supplementing, or eliminating many of these details.

In a standalone embodiment, a user may download or otherwise acquire program code and install if on a user device such as a smartphone or tablet computer. After the program is installed, when preparing to sleep the user may use the program to set an alarm to be awakened after a select duration of time, or at a select time by the clock. In embodiments, the user device may detect when the user is asleep and dreaming, such as by detecting user sleeptime rapid eye movement (REM) by tapping into existing auditory or sensory data from the device or may predict when the user is likely to be at an optimal state—within the range (approximately) 15 minutes of the time requested—by storing and analyzing data of the occurrence of REM and NREM states based on user history and input.

Upon awakening, the user may user their user device to record their dream, or at least as much of their dream as they can remember. In embodiments, the user may use a virtual or tangible keyboard to key in the dream information. In embodiments, the user may describe the dream orally, in which case the software may convert the oral recording to text.

In embodiments, in the case the subject does not remember their dream details at all, a similar analysis method may be applied by asking the subject for their emotional state at the moment of awaking (aka a “Mood Tracker”). In embodiments, the mood tracking feature may be incorporated into the process pursuant to dream entry, and may be then used in conjunction to provide the analysis engine emotionally contextual data.

The program may detect terms describing structural story elements in the dream, such as environment, location, feelings, actions, activity, and operating characters. In embodiments, the program may match said words with likely associative descriptive words or terms (such as “Dog is: A. Vicious, B. Loyal, C. Friendly” etc.) based on existing word libraries or databases (Datamuse is one example of such a database). In embodiments the program may match such words with terms that have occurred in earlier dreams by this dreamer and/or by the collectively analyzed data from the program's database. In embodiments, the terms may be looked up in a dream term dictionary.

In embodiments, the program may prompt the user for additional information. The prompts may be standard phrases such as “what else can you tell me?”, or may include words detected in the dream description, such as “what else can you tell me about the flying?”. In embodiments, the program may prompt the user for corelating or significant life experience occurring in the user's current waking life, such as “preparing for a big presentation at work”.

When the program has gathered as much information of the dream, mood, and or other user-entry as reasonably possible from the user, and has analyzed the dream content as described in the foregoing, the program may generate a dream content analysis. In embodiments, the analysis may include analysis of dream imagery, such as to determine the possible symbolic meaning of certain images and image sequences that appeared in the dream, and/or an analysis of the entire dream sequence. In embodiments, the analysis may include likely contextual correlation between a dreamer's real-life experiences (physical and emotional) and their emotional ramifications. In embodiments, the analysis may suggest potential techniques rooted in positive psychology, cognitive behavioral therapy, mindfulness, and similar practices. In embodiments, such suggestions may include a guided interactive process using audio, video, and journaling.

In embodiments, the recorded dream information may be stored for future review. In embodiments, aspects of the dream, the keywords, the suggested symbolic meanings, the entire dream sequence, and the like may be stored locally for use in future analyses.

In embodiments, the user device may be coupled to a network by any known wired or wireless interface. In such embodiment, the program code may be stored and run on remote processing and data storage resources, such as in a computing cloud or a server computer remote from the user device (hereinafter collectively “server”. In these embodiments, various screens may be sent by the server to the user device for display. For example, the screens may be sent as or in web pages and displayed in a web browser running on the user device.

In these “online” embodiments, a user may initially be required to submit various user information to be stored on the remote server. The user information may include name, age, residential information, payment information, and the like. The user information may also include relevant health-related and medical data, or the server may be configured to interface with one or more repositories of user data maintained by other entities that may be relevant to analyzing the user's dreams, such as medications, mental health history, and the like. The user information may also include relevant personal information regarding relationships, feelings, major life events, past experiences, field of work etc. The dream analysis may be performed by the server and provided to the user by the server.

Further, in online embodiments dream information from a plurality of users may be anonymized, aggregated, combined, analyzed, and stored for future use. For example, statistical analysis may be able to refine the meanings of certain terms, and imagery as experienced by dreamers. Using dream information from a plurality of users, perhaps even a multitude of users, it is contemplated that dream analysis may be refined and made more relevant to users. For example, in an embodiment the results of such analyses may be used to enhance and improve the analyses of users' dream aspects and sequences.

In embodiments the stored and analyzed information may be used to improve the solution and services to the multitude of users and to convey the multitude experiential information from the plurality of users and may be shared with users to illustrate the relation between the user's dream and other users' dream content.

In embodiments the “Big Data” database may manage, store, visualize, and analyze and generate insights from said database in real-time.

In embodiments said database may be applied via an application programming interface to sell, share, or otherwise inform organizations, companies, and individuals in the mental healthcare arena of patterns in the collective or psychographic group cognitive behaviors, or about patterns and movement in a specific user's profile if and when consent is set and encrypted sharing requested by the user.

In embodiments, a marketplace platform facilitating the matching, arranging and video conferencing of users with service providers such as coaches.

Referring now to the figures, FiGs.

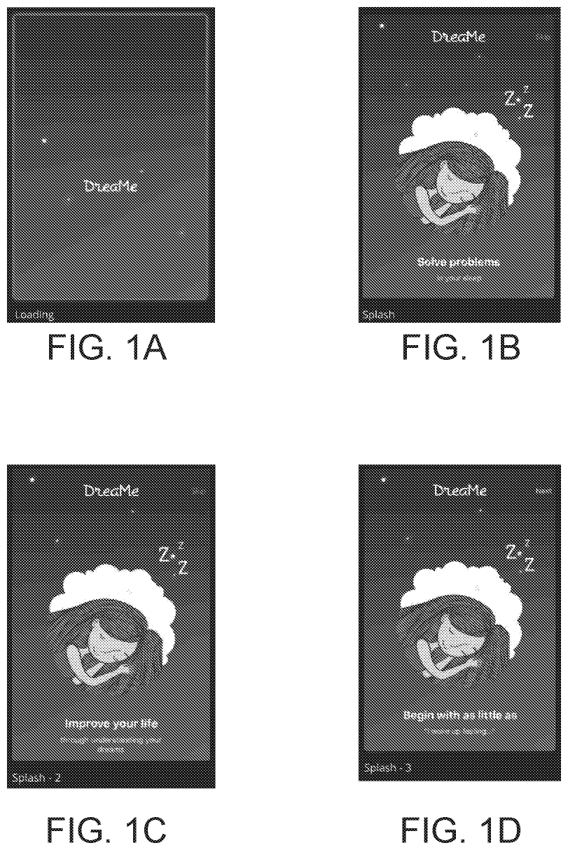

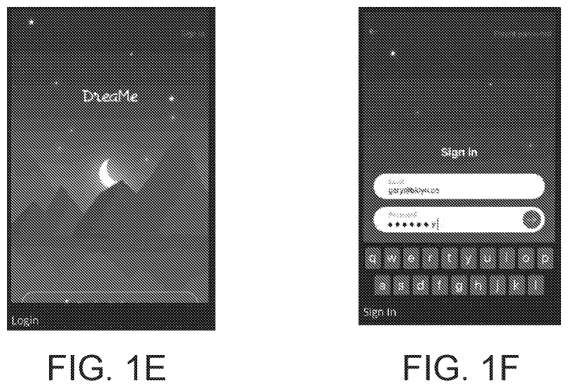

FIGS. 1A to IF show exemplary screenshots including an introductory screen, splash screens, and login screens.

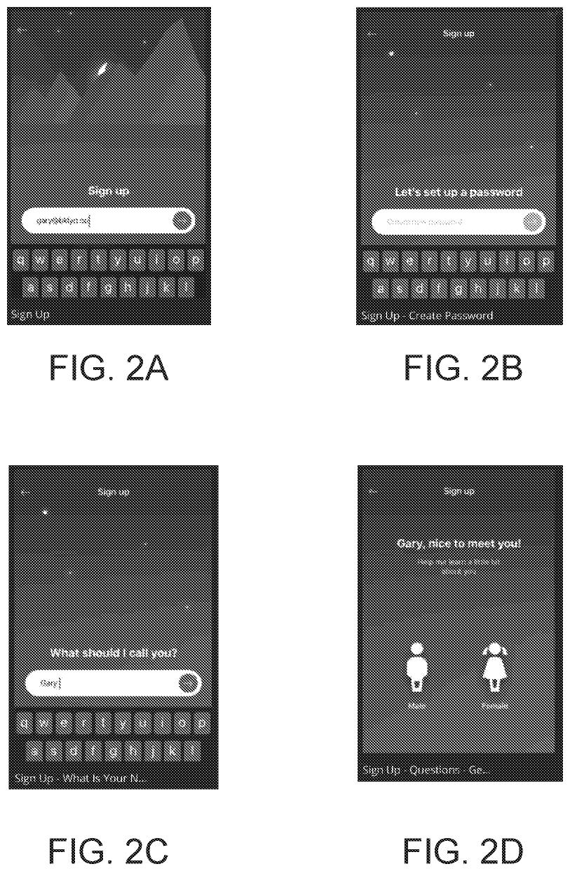

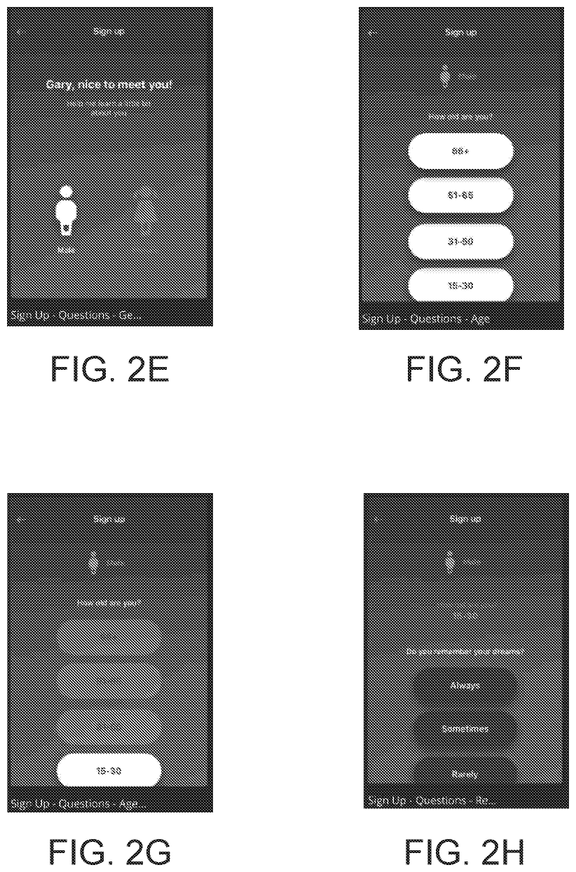

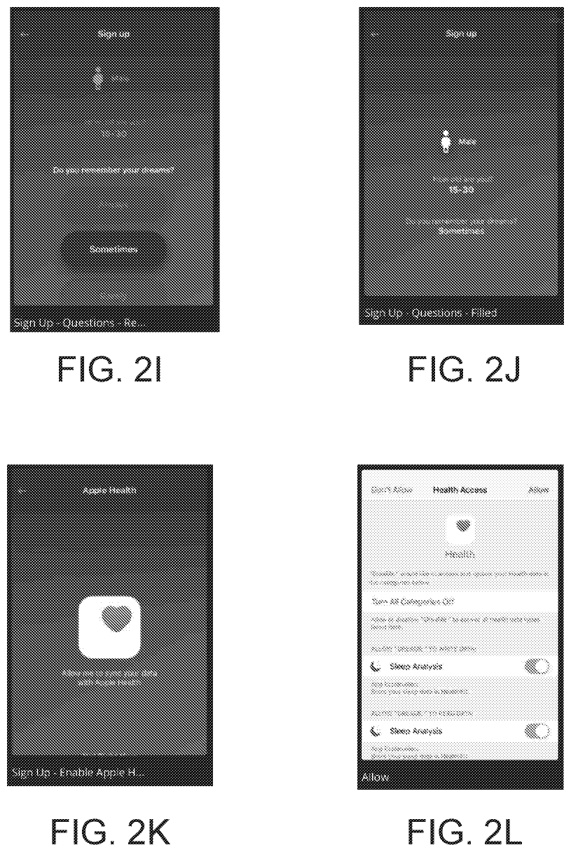

FIGS. 2A-2L show exemplary screenshots including screens to gather user information.

For example, a registration process may include setting up a username and password, for a subsequent login process to user the program. In addition, screens for collection user information such as gender, age range, previous dream experiences, and conditions that may affect dreams are shown.

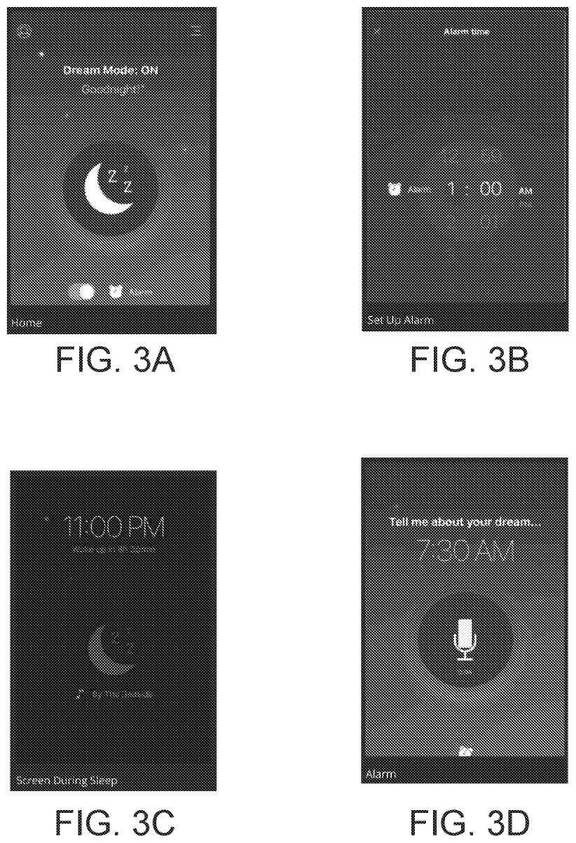

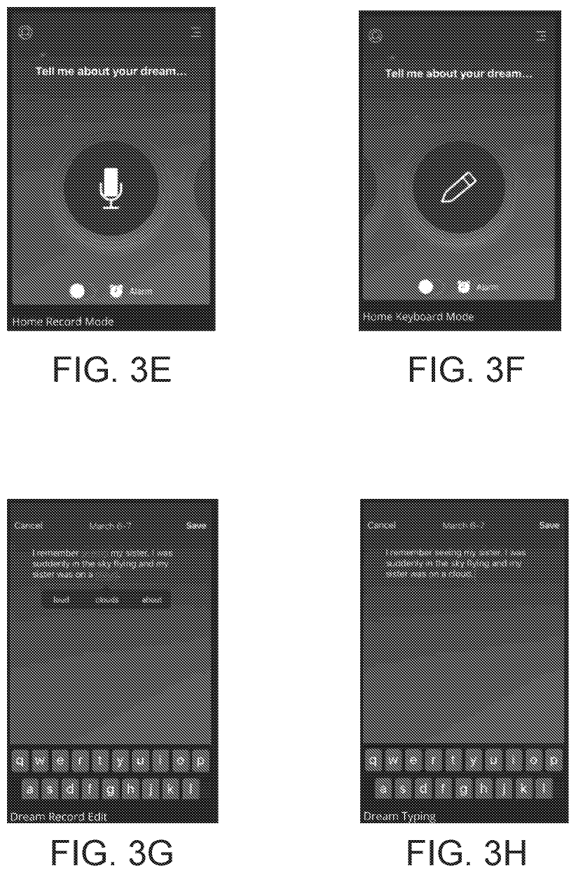

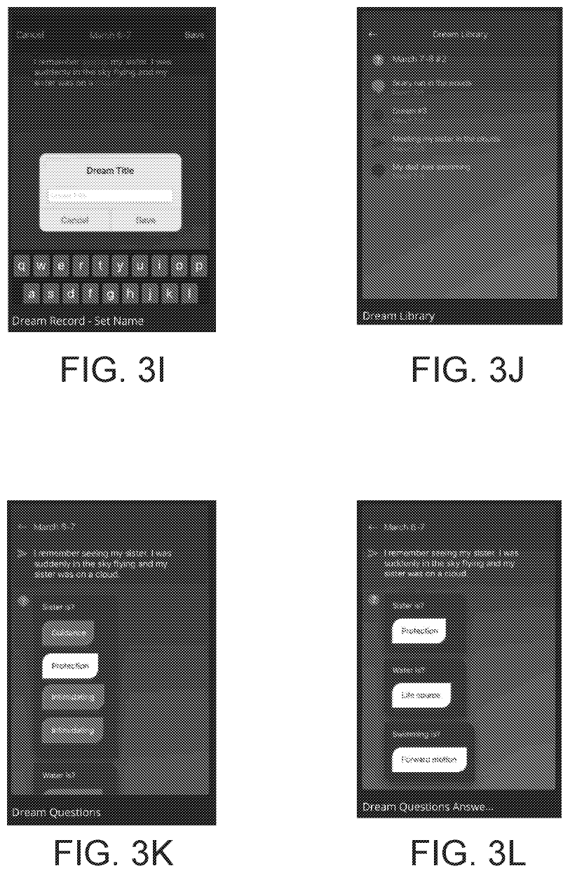

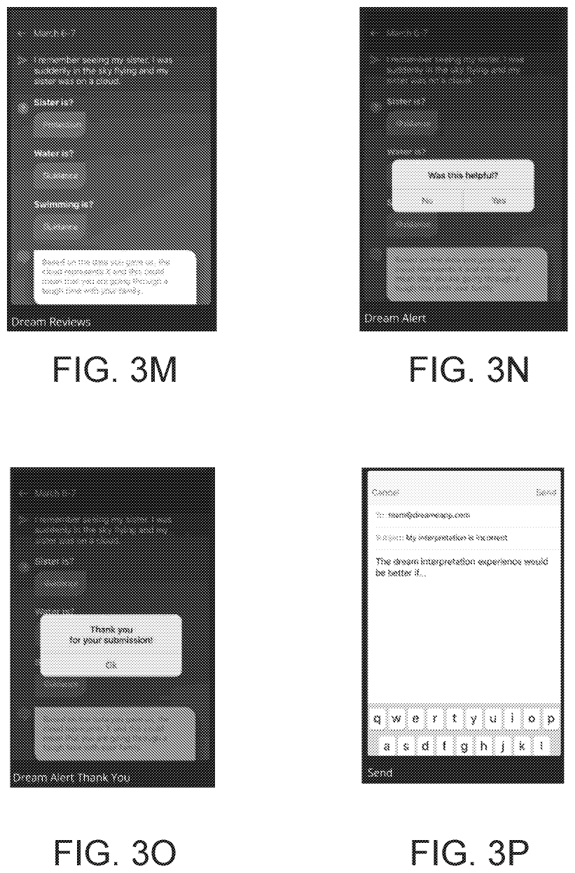

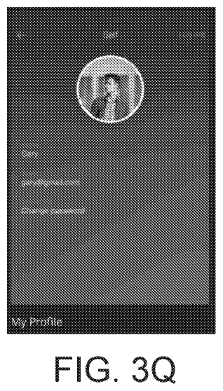

FIGS. 3A-3Q show exemplary screenshots including screens for setting a dreamtime alarm, gathering dream information, prompting for further information that may be useful for analyzeing the dream, and providing suggested analyses of salient dream elements and of the dream in general.

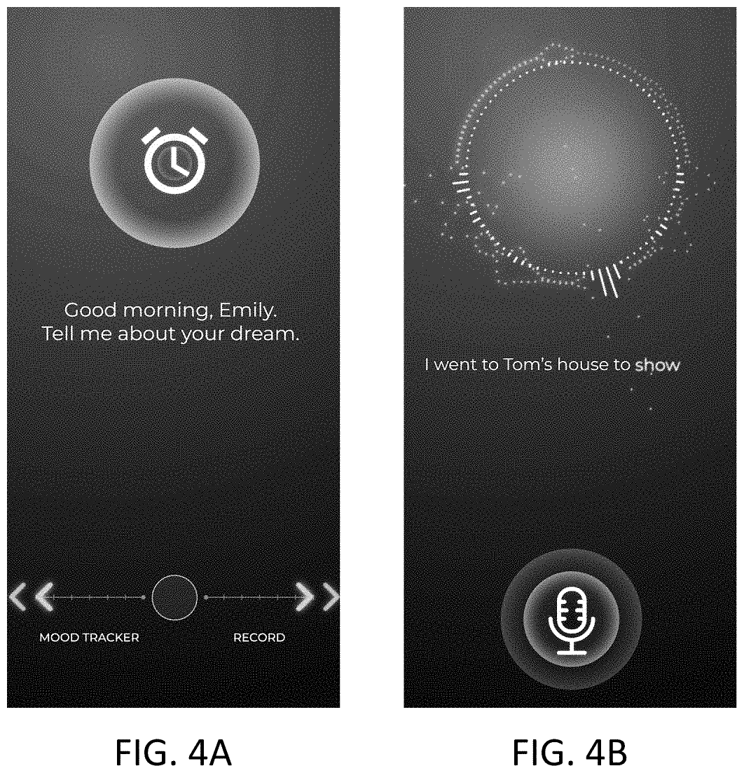

FIG. 4A shows exemplary screenshot of a screen for the alarm being activated at the previously selected time, prompting the user to begin awaking and select to either record the dream or, alternately, use the mood tracker to enter their mood.

FIG. 4B shows exemplary screenshot of a screen for the user to record the dream.

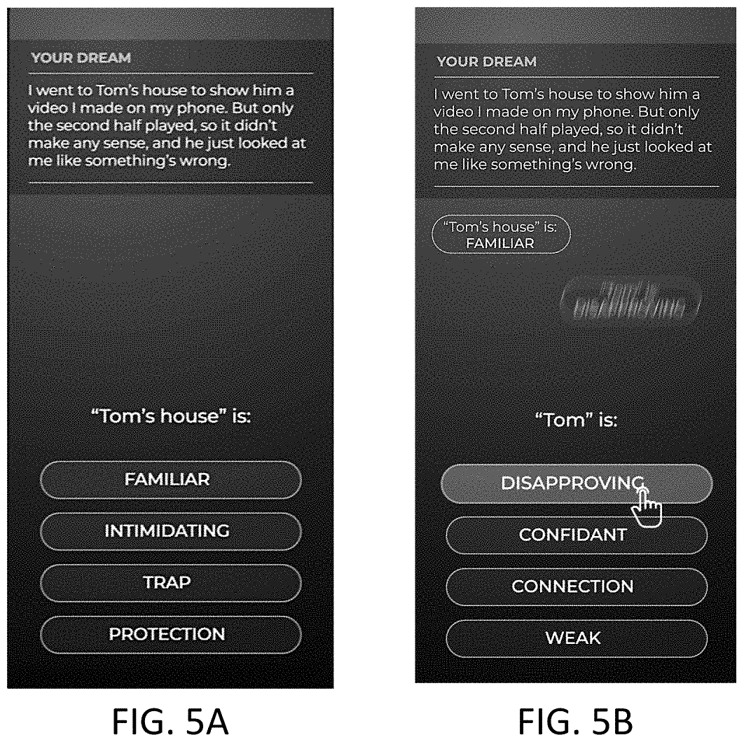

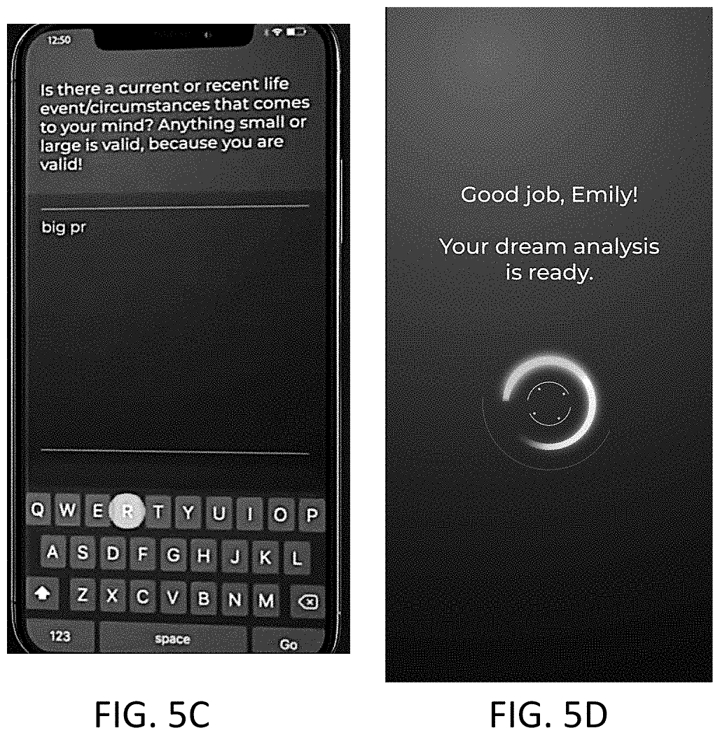

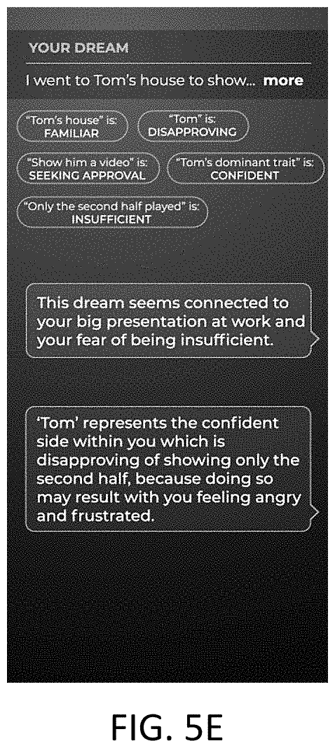

FIG. 5A shows exemplary screenshot of a screen for the recorded dream to display as the conversational agent prompts the user with questions regarding a dream element, and suggesting possible meaning for the user to select from.

FIG. 5B shows exemplary screenshot of a screen for the recorded dream, as well as the user's input from FIG. 5A to display, as the conversational agent prompts the user with questions regarding an additional dream element, and suggesting possible meaning for the user to select from.

FIG. 5C shows exemplary screenshot of a screen for prompting the user to type in any current life event or circumstances that seems related to the dream.

FIG. 5D shows exemplary screenshot of a screen displaying an animated symbol depicting the engine's analysis processing.

FIG. 5E shows exemplary screenshot of a screen for the dream text, the user's input from FIGS. 5A-5B, and additional similar inputs to display, as the conversational agent conveys the dream's analysis to the user in a conversational/textual manner.

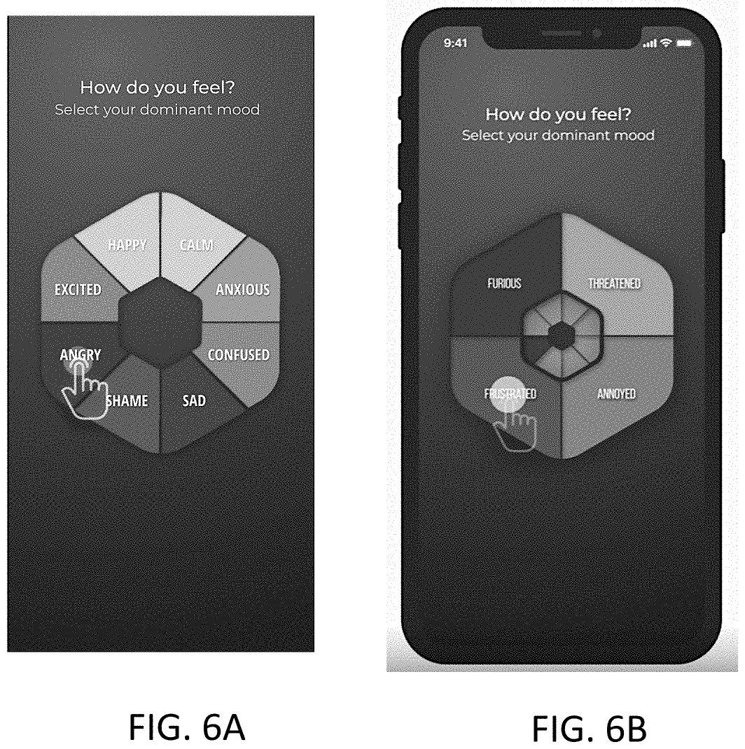

FIG. 6A shows exemplary screenshot of a screen displaying the top tier of the mood tracker as a color-coded diagram for the user to select their current mood.

FIG. 6B shows exemplary screenshot of a screen displaying the second tier of the mood tracker as a color-coded diagram displaying a number of sub-moods related to the user's first tier pick, for the user to select their current sub-mood.

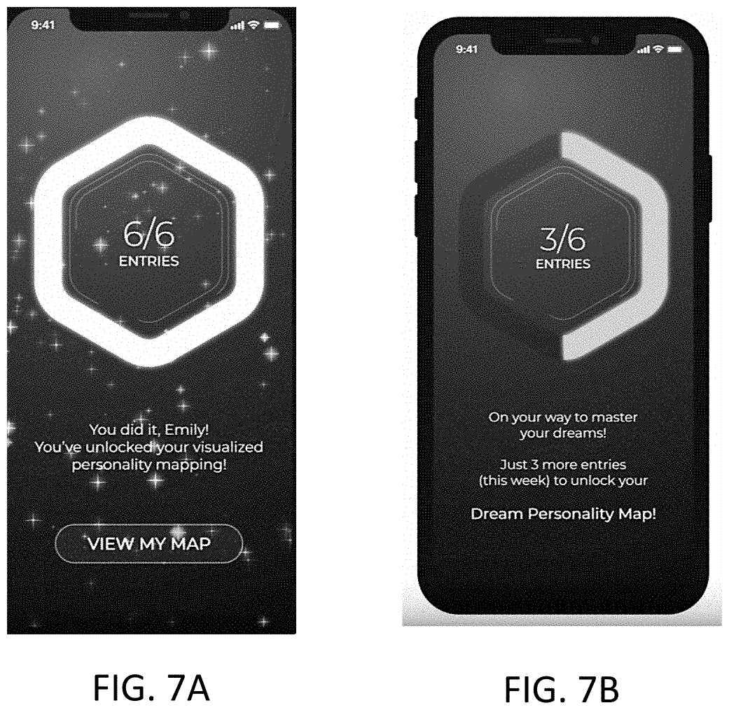

FIG. 7A shows exemplary screenshot of a screen displaying the number of the user's entries for the time period set by the engine as an encouraging “badge” for the user to see the points they earn towards a bonus reward.

FIG. 7B shows exemplary screenshot of a screen displaying the latest entry as reaching the number of entries for the time period required as an award “badge” celebrating the user's milestone.

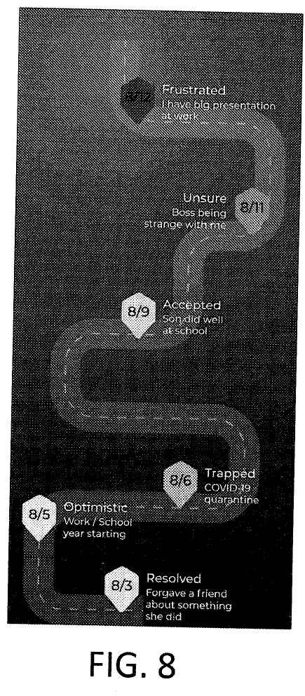

FIG. 8 shows exemplary screenshot of a screen for the awarded feature: A personalized map, diagram, or infographic displaying analyses as psychological thematic tagging in relation to the dates dreams were inputted and to the user's inputted correlating life events.

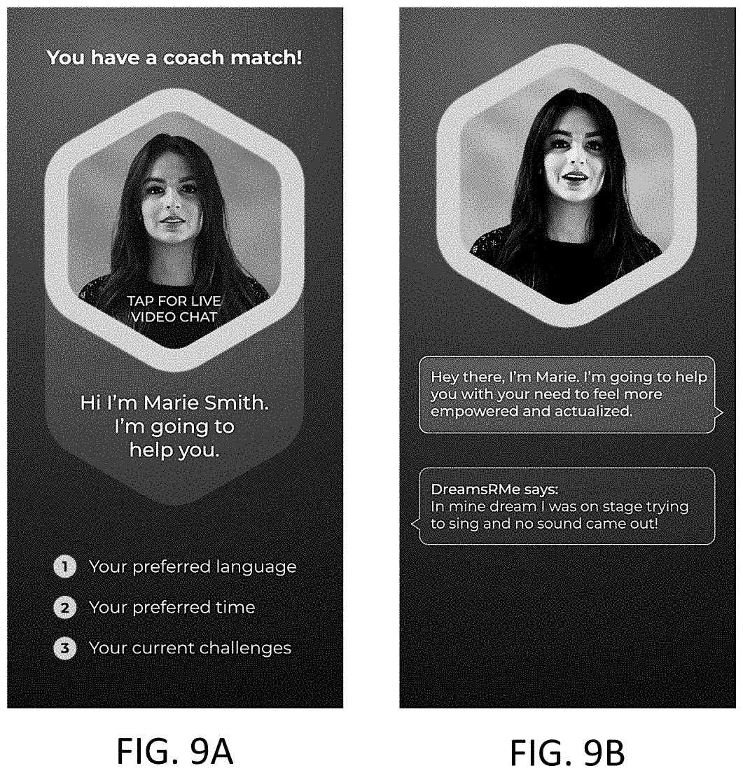

FIGS. 9A-9B show exemplary examples of a screenshot of the system.

A confidential academia dream study and analysis are included herewith as an appendix. The study contains information relevant to the development of the subject matter described in the foregoing.

Although the invention has been described and illustrated in exemplary forms with a certain degree of particularity, it is noted that the description and illustrations have been made by way of example only. Numerous changes in the details of construction, combination, and arrangement of parts and steps may be made without deviating from the scope of the invention. Accordingly, such changes are understood to be inherent in the disclosure. The invention is not limited except by the appended claims and the elements explicitly recited therein. The scope of the claims should be construed as broadly as the prior art will permit. It should also be noted that all elements of all of the claims may be combined with each other in any possible combination, even if the combinations have not been expressly claimed.

What is claimed is:

1. A dream content analysis platform, comprising:
a processor;
a program storage device operatively coupled to the processor, having instructions stored thereon which, when executed by the processor, cause functions to be performed;
a conversational artificial intelligence agent that communicates back and forth between user and said processor/program; wherein said agent prompts the user to record a dream that was ongoing when the alarm woke the user; wherein said agent may prompt the user to enter their mood upon waking; wherein said engine may prompt the user to input more information, associations, or memories related to elements from the user's initial input; wherein said agent may suggest potential associations for the user to choose from.
a recording engine configured to audio-record user input describing the dream or any elements, symbols, and related associations;
a transcriber converting said recording audio recording to text;
a dream content analysis engine and storage device operatively coupled to the processor, containing: a plurality of symbols known to appear in dreams, each symbol stored in association with at least one corresponding possible meaning, thematically categorized dreams and associations with the various dream elements, emotional and cognitive associations, and any other psychographic data deemed valid and valuable;
a processor tagging said user inputs with a variety of recognized psychological terms describing mental states, such as ‘fear of rejection’, ‘need for autonomy’, ‘desire to bond’;
an alarm that is configurable to awaken a user at a range within the user's desired awaking time, when the user is organically transitioning from deep sleep to awaking;
a mood tracking system and interface wherein the user may select their appropriate mood from a mood “wheel” or diagram;
A collection of techniques, practices, physical and mental activities; wherein said techniques may be matched and proposed to the user; and
a user interface comprising a plurality of screens to be shown on a user device having a display.

2. The platform of claim 1, wherein the user interface includes:
screens for introducing a user to the platform;
screens for gathering user information;
screens for use in awakening the user and recording dream information;
screens for prompting the user for information relevant to the dream;
screens for presenting to the user an analysis of the dream;
screens for cataloging dreams and their respective analyses;
screens for presenting to the user visualized illustrations of the user's dreams, symbols, themes, in relation to the progression of time;
screens for modifying the operation of the dream analysis engine;
screens guiding the user in said techniques, using visual, auditory, and/or video.

3. The platform of claim 1 comprising: A sleep data processor and interface for tracking the user's sleep quality and sleep cycle patterns;
an interface and screens wherein said data may be displayed to the user

4. The platform of claim 1 comprising:
a coaching services marketplace platform comprising:
a multitude of assigned coaches and other personal growth professionals;
a video conferencing engine wherein users may participate in small group and/or one-on-one talking sessions with said coaches.

5. The platform of claim 1 comprising:
a community platform wherein users may post dreams, analyses, comment, share, and read helpful materials; and
a processor matching dreams according to thematic or tagging categories;
an interface delivering community-driven insights; and screens delivering said insights to the user.

6. A Big Data engine; wherein, data from the multitude of users is anonymized, aggregated, combined, analyzed, and stored for future use. For example, said engine may deploy an interface exclusively to be sold to mental healthcare providers, clinicians, or researchers as a unique resource of deep insights into cognitive behavioral analytics.
